(19) United States
(12) Patent Application Publication
(10) Pub. No.: US 2018/0353402 A1
(43) Pub. Date: Dec. 13, 2018

(54) PHOTOSTABLE, BROAD-SPECTRUM SUNSCREEN COMPOSITIONS

(71) Applicant: Garth Fisher Beauty LLC, Beverly Hills, CA (US)

(72) Inventors: D. Garth Fisher, Beverly Hills, CA (US); Regina S. Lim, Port Orange, FL (US); Fred G. Rojas, Daytona Beach, FL (US)

(21) Appl. No.: 16/002,274

(22) Filed: Jun. 7, 2018

Related U.S. Application Data
(60) Provisional application No. 62/516,148, filed on Jun. 7, 2017.

(51) Int. Cl. A61K 8/35; A61K 8/37; A61K 8/9789; A61K 8/29; A61K 8/27

(52) U.S. Cl. CPC A61K 8/35; A61K 8/37; A61K 2800/5922; A61K 8/29; A61K 8/27; A61K 8/9789

(57) ABSTRACT

A photostable, broad-spectrum, sunscreen composition having an SPF of at least 30, preferably an SPF of at least 50, containing a synergistic anti-glycation complex consisting essentially of (i) an extract of a botanical ingredient in the genus Osmanthus, and (ii) an extract of Camellia sinensis; and optionally, but preferably, Butyrospermum parkii (shea) butter.

### CROSS-REFERENCE TO RELATED APPLICATIONS

The present application claims priority to a United States provisional patent application entitled “Anti-Glycation, Photostable, Broad-Spectrum Sunscreen,” having U.S. Ser. No. 62/516,148 and filed on Jun. 7, 2017, the disclosure of which is hereby incorporated by reference.

### FIELD OF INVENTION

The present invention relates to topical compositions that provide broad spectrum protection against ultraviolet radiation (from 290 nm-400 nm).

### BACKGROUND OF THE INVENTION

Sunscreens were first developed to protect against sunburn (which is caused primarily, but not entirely, by UVB radiation). Sun Protection Factor (SPF) is a clinical (in vivo) measurement of a sunscreen's ability to protect against sunburn. Sunscreens labeled with a higher SPF indicate that a person can remain in the sun for a longer period of time without developing sunburn.

SPF, however, is not a fully informative metric of photoprotection. Importantly, SPF does not measure UV-A, a form of ultraviolet radiation that penetrates the skin deeper than UV-B. Recognizing this limitation, a different metric—“broad spectrum”—was developed to provide a “label” that a sunscreen protects against both UVA and UVB radiation.

Use of a sunscreen labeled as “broad spectrum” with an SPF value of more than 15 is widely recognized by many authorities, including professional medical societies, the Surgeon General and FDA, as an important tool for skin cancer prevention.

Cumulative sun exposure is also associated with photoaging—notably, wrinkles and solar lentigines (“liver spots”)—and cancer. See, e.g., Jean L. Bolognia, “Aging Skin”, American Journal of Medicine, Vol. 98, No. 1, pages S99-S103 (1995). See also, Richard G. Glogau, “Physiologic and Structural Changes Associated with Aging Skin,” Dermatologic Clinics, Vol. 15, No. 4, pp. 555-559 (1997).

In the United States, two physical (mineral) sunscreens have been approved by the FDA—zinc oxide or titanium oxide; both reflect or scatter ultraviolet radiation. The four most common organic UV-filters used in over-the-counter sunscreens in the United States are (i) avobenzone (butyl methoxydibenzoylmethane), a UVA filter absorbing radiation between 320 nm and 400 nm, and (ii) oxybenzone, octocrylene, and octinoxate (octylmethoxycinnamate), all UVB filters that absorb radiation between 280 nm and 320 nm. Avobenzone provides the broadest UV-A absorbance of any chemical or physical sunscreen.

Broad spectrum, high SPF sunscreens are not without concerns. Avobenzone is photolabile, which reduces its ability to effectively absorb UV radiation over extended periods of time. See, Afonso, S. et al. “Photodegradation of avobenzone: stabilization effect of antioxidants.” J. Photochem. Photobiol. B., Vol. 140, pp. 36-40 (2014); and Beasley, D G. et al. “Characterization of the UVA protection provided by avobenzone, zinc oxide, and titanium dioxide in broad-spectrum sunscreen products.” Am J Clin Dermatol Vol. 11, No. 6, pp. 413-21 (2010). See also, Kumar P, D. A. et al., “Patent review on photostability enhancement of avobenzone and its formulations.” Recent Pat Drug Deliv Formul, Vol. 9, No. 2, pp. 121-8 (2015).

It has also been reported that avobenzone can enhance the degradation of UVB protection agents in sunscreens. Lebwohl, M., et al. “Effects of topical preparations on the erythemogenicity of UVB: implications for psoriasis phototherapy.” J. Am. Acad. Dermatol. Vol. 32:469-71 (1995).

To help stabilize avobenzone, and reduce reactive oxygen species, antioxidants, such as vitamin C, E and ubiquinone, are incorporated into sunscreen formulations. See, e.g., Hanson, K. M. et al., “Antioxidants in sunscreens for improved ROS photoprotection,” Cosm. Toil., Vol. 126, pp. 712-716 (2011); see also, http://www.lorealparisusa.com/ingredient-library/avobenzone.aspx.

Cautions have also been raised about UV-B filters. For example, the presence of octocrylene, octylmethoxycinnamate, and benzophenone-3 for extended periods of time have been reported to produce UV-induced Reactive Oxygen Species (ROS). See, Hanson, K M et al, “Sunscreen enhancement of UV-induced reactive oxygen species in the skin” Free Radical Biology & Medicine, Vol. 41, pp. 1205-1212 (2006). See also, research findings from laboratory of Prof. C J Bardeen, University of California at Riverside, published at http://www.faculty.ucr.edu/˜christob/UVFilters6.html.

Some sunscreen active ingredients have been reported to be absorbed through the skin. See, e.g, Hayden, C G et al., “Systemic absorption of sunscreen after topical application. Lancet, Vol. 350, pp. 863-864 (1997); Jiang, R. “Absorption of sunscreens across human skin: an evaluation of commercial products for children and adults” Br. J. Clin. Pharmacol. Vol. 48, No. 4, pp. 635-637 (1999). Particular concerns have been raised about Benzophenone-3, which has been detected in human urine and breast milk. See, e.g., Walters, K A and M S Roberts, “Percutaneous absorption of sunscreens.” in Bronaugh, R L and HI Maibach, eds., Topical Absorption of Dermatological Products, pp. 465-481 (New York: Dekker; 2002); see also, Hany, J and R Nagel, “Detection of sunscreen agents in human breast milk” Dtsch. Lebensm. Rundsch. Vol. 91, pp. 341-345 (1993).

In a recent report on the Sunscreen Innovation Act, Theresa M. Michele, M.D., Director of FDA's Center for Drug Evaluation and Research's Division of Nonprescription Drug Products, commented that dermal absorption of sunscreens is a “significant discovery that needs to be considered.” See, “From Our Perspective: Helping to Ensure the Safety and Effectiveness of Sunscreens” (Nov. 22, 2016) accessed on Jan. 25, 2016 at http://www.fda.gov/Drugs/NewsEvents/ucm473752.htm.

Collagens are the primary proteins in mammalian connective tissue. Collagens make human skin stronger, thicker and more elastic, and are largely responsible for the smooth appearance in younger skin. Collagens begin as procollagen molecules comprised of three chains: two pro-α1(I) chains, produced by the COL1A1 gene, and one pro-α2(I) chain, produced by the COL1A2 gene. As the procollagen peptide is processed to form a mature collagen protein, the propeptide portion is cleaved off (type I C-peptide). Levels of type I C-peptide reflect the amount of collagen synthesized and can be assayed via an enzyme-linked, immunosorbent assay (ELISA).

After procollagens are processed, the resulting mature collagen molecules arrange themselves into long, thin fibrils. Cross-linking of collagen molecules, in turn, results in strong Type I collagen fibrils. See, http://ghr.nlm.nih.gov/gene=collal. Collagen types I and III are the major structural components in the skin, accounting for over 70% and 15%, respectively, of the dry weight of skin and providing the dermis with tensile strength and stability. Collagen type IV is responsible for the mechanical stability of the skin's scaffolding, which connects the epidermis and dermis.

Maintenance of firm, young-looking skin, requires a balance between collagen synthesis and degradation. With aging, collagen turnover slows, collagen levels drop and the collagen macromolecules undergo significant physicochemical changes, including decreased solubility, elasticity, and sensitivity to protease digestion. See, e.g., Schnider, S. L. and R. R. Kohn, “Effects of Age and Diabetes Mellitus on the Solubility and Nonenzymatic Glucosylation of Human Skin Collagen” J. Clin. Invest. Vol. 67, pp. 1630-1635 (1981).

Glycation, non-enzymatic bonding of a protein or lipid molecule with a sugar molecule, and enzyme-mediated glycoxylation contribute to the modification of collagen in aging skin. The initial product of protein glycation of protein, fructoselysine (FL), may form the lysine-arginine cross-link, pentosidine. Glycated proteins react further, especially via oxidative reactions, leading to the formation of late-stage Maillard products. FL may be cleaved oxidatively to form Nf-(carboxymethyl) lysine (CIVIL). Glycated hydroxylysine may yield N4-(carboxymethyl) hydroxylysine (CMhL). Collectively, these advanced glycosylation end products are described in the literature and in the present application as “AGE products”. See, e.g., Dyer, D G et al. “Accumulation of Maillard reaction products in skin collagen in diabetes and aging,” J. Clin. Invest. Vol. 91, pp. 2463-2469 (1993).

Glycation promotes the formation of free radicals and exacerbates oxidative damage to proteins and lipids. See, e.g., Yan S D et al., “Enhanced Cellular Oxidant Stress by the Interaction of Advanced Glycation End Products with Their Receptors Binding Proteins” J. Biol. Chem., Vol. 269, No. 13, pp. 9889-9897 (1994). AGE products themselves are also linked to formation of oxygen free radicals. See, e.g., T J Lyons et al., “Decrease in Skin Collagen Glycation with Improved Glycemic Control in Patients with Insulin-dependent Diabetes Mellitus,” Vol. 87, pp. 1910-1915 (1991).

Accumulation of AGEs in cells affects extracellular and intracellular structure and function, including by forming crosslinks in the basement membrane of the extracellular matrix and interacting with RAGE, a signal transduction receptor of AGE products, primarily via glycoproteins which have been modified through Maillard-type reactions. Moreover, once formed, AGE products have been reported to induce crosslinking of collagen, even in the absence of sugars and oxygen. Sajithlal G B, “Advanced Glycation End Products Induce Crosslinking of Collagen In Vitro” Biochim Biophys Acta. Vol. 1407, No. 3, pp. 215-24 (1998).

Activation of RAGE, in turn, causes inflammation and oxidative stress, further contributing to skin aging. Ramasamy R, “Advanced glycation end products and RAGE: a common thread in aging, diabetes, neurodegeneration, and inflammation” Glycobiology, Vol. 15, No. 7, pp. 16R-28R (2005).

Glycated collagen fibers have reduced regenerative ability, leading to the wrinkles, creping, and sagging that characterize skin aging. These visible signs of aging (e.g., fine lines and wrinkles) are correlated not only with decreased synthesis of collagen but increased enzymatic degradation of collagen by collagenases, in particular Collagenase I, also known as Matrix Metalloprotease 1 (MMP1). MMP-1 is a zinc and calcium dependent endopeptidase that is produced and released from both dermal fibroblasts and keratinocytes. Levels of both active and inactive MMP-1 can be assayed using a fluorescence-based ELISA.

The degradative activity of MMP1 is regulated by the concentration of an endogenous protease inhibitor, Tissue Inhibitor of Matrix Metalloprotease-1 (TIMP1). When cells undergo glycation, synthesis of MMPs increases, and levels of MMP inhibitors decrease. H. Pageon, “Collagen glycation triggers the formation of aged skin in vitro”, Eur. J. Dermatol., Vol. 17, No. 1, pp. 12-20 (2007). Relatedly, glycation has been associated with cellular senescence, a state in which fibroblasts switch from matrix-producing to matrix-degrading. See, e.g., “Advanced Glycation End Products Enhance Expression of Pro-apoptotic Genes and Stimulate Fibroblast Apoptosis through Cytoplasmic and Mitochondrial Pathways” J. Biol. Chem. Vol. 280, No. 13, pp. 12087-95 (2005) and modification of Collagen IV. HM Raabe, “Biochemical alterations in collagen IV induced by in vitro glycation” Biochem. J., Vol. 319, pp. 699-704 (1996).

Polyphenols are associated with reducing and inhibiting UV-induced expression of matrix metalloproteinases (MMP-2, MMP-3, MMP-7, and MMP-9), as well as UV-induced photo-oxidation, which produces reactive oxidation species.

DermalRx® LuShield™ available from Biocogent LLC (Stony Brook, N.Y.) is an extract of flowers of Osmanthus fragrans. The supplier's technical data sheet describes the product as providing protection from AGE (Advanced Glycation End-products) which results in “fibroblast collapse,” a condition characterized by “loss of integral extracellular matrices.”

US patent application Pre-Grant Publication 2015/0104399 assigned to Biocogent LLC is directed to skin care compositions comprising extracts of plants of the genus Osmanthus; and methods of reducing or preventing fibroblast collapse, reducing or preventing glycation, maintaining or increasing skin elasticity and firmness and reducing the appearance of aging by topical application of the compositions containing Osmanthus extracts. Additional components that can be combined with Osmanthus extracts are described only generally in terms of broad categories of skincare ingredient selected from emollients, vitamins, lipids, sunscreen agents, moisturizers, exfoliating agents, surfactants, solvents, colorants, maskants, fragrances and preservatives. PG-Pub 2015/0104399 does not disclose any specific sunscreen ingredient, much less the specific ingredient combinations used in practicing the methods of the present invention.

Japanese Patent Application Publication No. JP 2005-126368 teaches topical compositions containing extracts of Osmanthus fragrans and/or Mangolia liliflora, and the use of those compositions for “whitening” (i.e., lightening) skin by inhibiting the generation of melanin.

Scavenox™ GTE (Green Tea Extract) is described in a technical data sheet (TDS) from its supplier, Biogent LLC, as a proprietary concentrate of the active anti-oxidant and anti-inflammatory components of green tea including polyphenols and epigallocatechin gallate (EGCG) made by “high energy sonic jet process technology”.

Among the many techniques known for extracting active constituents from plant biomass are ultrasound-assisted extraction. See, e.g., “Extraction and isolation of catechins from tea” J. Separation Sci. Vol. 33, pp. 3415-3428 (2010) citing Mason, T. et al., “The uses of ultrasound in food technology” Ultrason. Sonochem. Vol. 3, pp. S253-S260 (1996).

The technical data sheet for Scavenox™ GTE describes its use for inhibition of oxidative stress, prevention of damage to extracellular matrix proteins caused by elastase and matrix metalloproteinases, and inhibition of phospholipase-2 activity. The potential role of phospholipase-2 inhibitors in treating inflammatory skin diseases is discussed by Dan et al., in Eur. J Pharmacol. Vol. 691 (1-3), pp. 1-8 (2012).

U.S. Pat. No. 5,306,486, now expired, teaches a “sunscreen cosmetic” comprising:

(i) from about 0.001 to about 20% by weight of green tea and (ii) from about 0.001 to about 25% by weight of a sunscreen compound which is effective to at least partially block ultraviolet radiation from harming human skin.

U.S. Pat. No. 8,435,541 describes a topical composition comprising: (i) an MMP inhibitor—namely, Glycine soja (soybean) seed extract; (ii) a tissue inhibitor of metalloproteinase (“TIMP”)—namely, acetyl hexapeptide-20; and (iii) at least one natural extract selected from the group consisting of Tremella fuciformis (snow fungus) extract; Eugenia caryophyllus (clove) extract; Camellia sinensis (green tea) extract (including Scavenox™ GTE sold by Biocogent, Ltd.); Pyrus malus (apple) fruit extract, Cinnamomum cassia (cinnamon) bark extract, Rheum palmatum (Chinese rhubarb) extract, and combinations thereof.

Increases in trans epidermal water loss (TEWL) are reported with one minimal erythema dose (MED) of UV irradiation. See, Lim S H, et al., “Change of biophysical properties of the skin caused by ultraviolet radiation-induced photo-damage in Koreans” Skin Res. Technol. Vol. 14, No. 1, pp. 93-102 (2008).

It is well-known in the dermatological arts that avobenzone undergoes photochemical degradation and is “photounstable”. The uses of octocrylene, a sunscreen filter that absorbs ultraviolet radiation in the range 290-320 nm, and anti-oxidants, are described in the literature. See, e.g., Kumar P et al., “Patent review on photostability enhancement of avobenzone and its formulations.” Recent Pat Drug Deliv Formul. Vol. 9, No. 2, pp. 121-8 (2015); Afonso, S. et al., “Photodegradation of avobenzone: stabilization effect of antioxidants” J Photochem. Photobiol. B. Vol. 140, pp. 36-40 (2014).

There has been, and remains, a long-felt, but unmet need for a sunscreen composition that is photostable, broad-spectrum and reduces and/or prevents accumulation of advanced glycosylation end products in the skin. That need is met by the topical compositions of the present invention.

### BRIEF DESCRIPTION OF THE DRAWINGS

FIG. 1 shows the absorbance of a sunscreen composition of the present invention across the UV spectrum (from 290 nm to 400 nm), both before (“initial”) and after 18 MEDs of irradiation from a solar simulator.

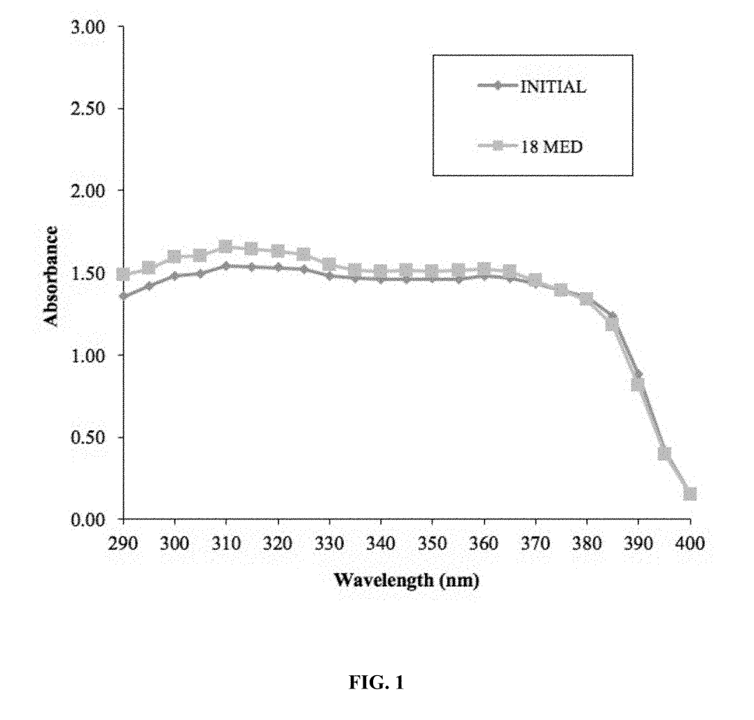

### SUMMARY OF THE INVENTION

Methods of reversing formation of and preventing accumulation of advanced glycation end products (AGEs) in skin comprising the step of applying to the skin a sunscreen composition having an SPF of at least 15, preferably an SPF of at least 30, and still more preferably an SPF of at least 50, and containing a synergistic anti-glycation complex consisting essentially of, and in preferred embodiments consisting of, an extract of a botanical ingredient in the genus Osmanthus, preferably Osmanthus fragrans, and an extract of Camellia sinensis. The synergistic anti-glycation complex may, and in certain preferred embodiments, does contain Butyrospermum parkii (shea) butter.

### DETAILED DESCRIPTION OF THE INVENTION

“Minimal erythema dose” (“MED”) means the quantity of erythema-effective energy (expressed as Joules per square meter) required to produce the first perceptible, redness reaction with clearly defined borders.

“Sun protection factor” (“SPF”) value is defined by the formula:

$$\frac{MED ( protected skin - with sunscreen applied )}{MED ( unprotected skin - without sunscreen applied )}$$

As used in the present application, by the term “photostable” is meant a sunscreen whose UVA/UVB absorbance ratio (measured in terms of normalized area under the curve) does not change by more than 10% between (a) an initial measurement and (b) measurement after exposure to UV radiation equivalent to a dose of 18 MEDs. Sunscreen is applied at a concentration of 2 mg/cm² to VITRO-SKIN®. The source of UV radiation is a solar simulator, most commonly a Xenon Arc solar simulator, that can produce light spectra meeting the SPF testing criteria set by the United States Food and Drug Administration, the European Cosmetic Toiletry and Perfumery Association, the Japanese Cosmetic Industry Association and other regulatory bodies. VITRO-SKIN® is a testing substrate available from IMS, Inc. (Portland, Me.) that mimics the topography, pH, critical surface tension, ionic strength and certain aspects of the chemical reactivity of human skin (e.g., sunless tanning from exposure to DHA and erythrulose). Use of VITRO-SKIN® in testing the efficacy of sunscreen formulations is described in numerous patent publications, including U.S. Pat. No. 9,149,664, U.S. Pat. No. 7,799,317, and U.S. Pat. No. 7,033,577, the disclosures of which are incorporated herein by reference, and has been reported at scientific symposia, including the Proceedings of 20^{th} Congress of the International Federations of Cosmetic Chemists. See, http://ims-usa.com/vitro-skin-substrates (accessed on Jun. 6, 2018).

The topical compositions of the present invention contain a Synergistic Anti-Glycation (“SAG”) Complex consisting essentially of, and in preferred embodiments consisting of, (i) an extract of a first botanical ingredient in the genus Osmanthus, preferably Osmanthus fragrans, that reduces or prevents accumulation of Advanced Glycosylation End (AGE) products in the skin and (ii) an extract of a second botanical ingredient that is a varietal of Camellia sinensis, preferably Camellia sinensis var. sinensis or Camellia sinensis var. assamica, in a combined amount sufficient to reduce the formation of Reactive Oxygen Species and/or reduce inflammation in skin.

In preferred embodiments of the invention, in which the first botanical ingredient is Osmanthus fragrans, the extract contains at least one triterpene (or derivative thereof), and a flavonoid complex that contains at least two of a flavonol, a flavan-3-ol (also known as flavanol), a flavone, a flavanone and derivatives thereof, including glycosides.

The at least one triterpene (or derivative thereof) is preferably lupeol.

The flavonoid complex preferably contains: quercetin and kaempferol, both flavanols; isoscutellarein, an apigenin flavone; and narigenin, a flavanone.

In preferred embodiments of the invention, the varietal of Camellia sinensis contains epigallocatechin gallate (EGCG), an ester of epigallocatechin and gallic acid that conforms to the structure:

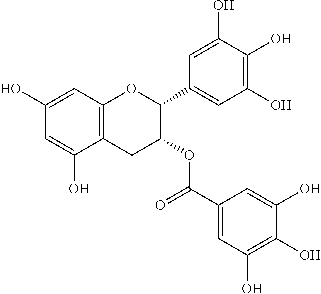 [SMILES: O=C(O[C@@H]1Cc2c(O)cc(O)cc2O[C@@H]1c1cc(O)c(O)c(O)c1)c1cc(O)c(O)c(O)c1]

In one set of embodiments, the topical compositions of the present invention are sunscreens that contains UVA and UVB filters, each as described below.

UV-A filters absorb ultraviolet rays with wavelengths in the range of 320 to 400-nm, which penetrate more deeply into the skin and can cause cancer. As a UV-A filter, topical compositions of the present invention contain avobenzone, preferably at a concentration of from about 1% to about 3%, and most preferably at a concentration of about 3%. Unless otherwise noted, all percentages are the amount/concentration of the ingredient on a weight/weight basis of the total composition.

UV-B filters absorb ultraviolet rays with wavelengths between 290 nm and 320 nm, which are the primary cause of sunburn. Topical compositions of the present invention contain at least one UV-B filter. One especially preferred UV-B filter is octrocrylene, which is preferably present in compositions of the present invention at a concentration of from about 5% to about 10%, and most preferably at a concentration of about 5%.

In preferred embodiments, in addition to octocrylene, topical compositions of the present invention contain a second UV-B filter, preferably a salicylate derivative.

Still more preferably, topical compositions of the present invention contain two salicylate derivatives that absorb UV-B radiation—homosalate and octisalate.

The two salicylates are preferably present in compositions of the present invention at a combined concentration of at least about 10%. More preferably, the two salicylates are present in compositions of the present invention are present such that the ratio of homosalate to octisalate is about 2:1.

The topical compositions of the present invention have an SPF of at least about 15, preferably an SPF of at least 30, and still more preferably an SPF of at least 50, where SPF is determined in accordance with Section 201.327(j) of Title 21 of the Code of Federal Regulations. More particularly, sunscreen products of the invention are applied at a concentration of 2 mg/cm² to subsites on a subject's back, between the beltline and the shoulder blades (scapulae) and lateral to the midline. A finger cot is used to spread the sunscreens (of the invention and FDA SPF standard) as evenly as possible. Fifteen minutes after applying the sunscreen products, a series of UV radiation doses are exposed to test subsites.

The source of UV radiation is a single port or multiport solar simulator filtered to provide a continuous emission spectrum from 290 to 400 nanometers (nm) with erythema-effective radiation distributed in each of the following wavelength ranges:

| Wavelength | Percent erythemal |
|---|---|
| range (nm) | contribution |
| <290 | <0.1 |
| 290-300 | 1.0-8.0 |
| 290-310 | 49.0-65.0 |
| 290-320 | 85.0-90.0 |
| 290-330 | 91.5-95.5 |
| 290-340 | 94.0-97.0 |
| 290-400 | 99.9-100.0 |

Erythema action spectrum is determined in accordance with 21 CFR § 201.327(i)(1)(ii) and ISO Standard 17166 CIE S 007/E entitled “Erythemal reference action spectrum and standard erythema dose,” (Nov. 15, 2000).

Each dose is 25 percent greater than the previous dose. After the UV doses are administered, immediate responses (darkening/tanning or reddening) are recorded. The exposed areas are then shielded from further UV radiation for 16 to 24 hours, at which time minimal erythema dose (MED) is determined. MED is the smallest UV dose that produces perceptible redness of the skin (erythema) with clearly defined borders at 16 to 24 hours after UV exposure.

Sunscreen compositions of the present invention provide “broad spectrum” protection over the UV spectrum from 290 to 400 nm, by which is meant the sunscreens have a mean critical wavelength of 370 nm or greater. Critical wavelength is the wavelength at which the integral of the spectral absorbance curve is 90 percent of the integral over the UV spectrum from 290 to 400 nm. Determination of “broad spectrum” is made in accordance with Section 201.327(j) of Title 21 of the Code of Federal Regulations.

Without wishing to be bound by a theory, Applicant believes the high photostability of the topical compositions of the present invention is attributable to the SAG Complex, which provides an antioxidant reservoir that deactivates the ROS (within the strata granulosum, spinosum, and basale) generated by the UV photons that the sunscreens do not absorb in the stratum corneum.

The topical compositions of the present invention are formulated in a cosmetically acceptable vehicle which can be a cream, ointment, or a lotion that can be an emulsion. Cosmetically acceptable vehicles are well known in the art. Some are described in, for example, PP Gerbino, ed., Remington: The Science and Practice of Pharmacy (2006).

The cosmetically acceptable vehicle may also include additional ingredients, for example, anti-inflammatory agents, short-chain peptides, including lipopeptides that increase the expression of genes that code for collagen and elastin and/or decrease the expression of genes that code for collagenases (including matrix metalloproteases). It is critical that any additional ingredients not interfere with the ability of the SAG Complex to reduce and prevent the accumulation of dermal AGEs.

The topical compositions of the present invention include 0.05% to 5.0%, preferably 0.1% to 2.0%, by weight, based on the total weight of the composition, of the SAG Complex.

The SAG Complex may, and in preferred embodiments, does contain Butyrospermum parkii (shea) butter

The remainder of the composition may be, and preferably is comprised of: (i) a silicone (or a derivative thereof), and/or (ii) a mixture butters comprising Butyrospermum parkii (shea) butter and at least two, preferably at least three, and still more preferably at least four additional butters selected from the group consisting of Shorea robusta seed butter; Elaeis guineensis (palm) butter; Theobroma cacao (cocoa) seed butter; Bassia latifolia seed butter; Mangifera indica (mango) seed butter; Garcinia indica seed butter; Illipe butter; Theobroma grandiflorum seed butter; Astrocaryum murumuru seed butter; and Astrocaryum tucuma seed butter; and/or (iii) an antioxidant vitamin, preferably Vitamin E (or a derivative thereof).

As used in the present application, the term “butter” is to be understood to mean a class of triglyceride-based materials (e.g., oils) having a melting point from about 20 to about 40° C. One method of making butters is hydrogenation, a process of reacting the double bonds in unsaturated triglycerides with hydrogen. Butters can also be created through esterification reactions.

In one particularly preferred embodiment, the topical compositions of the present invention contain silicone at a concentration of greater than about 1%. One preferred silicone is dimethicone with a viscosity of from 20 cst to 30,000 cst.

Optionally, in certain embodiments, the broad-spectrum, sunscreen composition of the present invention contains one or more film-forming polymers that make the sunscreen “water resistant”, and still more preferably “very water resistant” as those terms are defined in 21 CFR § 352.76.

Non-limiting examples of film-forming polymers include the following: waterborne polyurethane dispersions, for example polyurethane-34 available from Bayer Material Science under the tradename Baycusan™ C1000; cyclodextrin in combination with a mixture of dimethicone and vinyldimethyl/trimethylsiloxysilicate/dimethicone crosspolymer, available, respectively as Cavamax™ and Belsil REG 1100™, from Wacker Chemical Corp; VP/dimethiconylacrylate/polycarbamyl polyglycol ester/VP/polycarbamyl polyglycol ester available as Pecogel™ HS-501 from Phoenix Chemicals Inc; cyclopentasiloxane (and) diphenyl dimethicone available as Gransil™ C-DPDM from Grant Industries; grafted silicones (e.g., C₂₀₋₄₀ alkyl dimethicone); polyvinylpyrrolidone (PVP) derivatives, e.g., triacontanyl PVP available under the tradename Ganex™ from Ashland.

The SAG Complex may also be included in a broad-spectrum mineral sunscreen, one containing titanium dioxide (TiO₂) and/or zinc oxide, preferably both TiO₂ and ZnO.

In preferred embodiments, the broad-spectrum mineral sunscreen containing ZnO, TiO₂ and the SAG is further comprised of Butyrospermum parkii (shea) butter.

In preferred embodiments, the broad-spectrum sunscreen of the present invention does not contain oxybezone.

In still other preferred embodiments, the broad-spectrum sunscreen of the present invention does not contain octinoxate.

In certain even more preferred embodiments, the broad-spectrum sunscreen of the present invention does not contain oxybenzone or octinoxate.

### EXAMPLES

The present invention is further described by reference to the following examples. These examples are provided for purposes of illustration only, and are not intended to be limiting. It is to be understood that numerous modifications may be made to the illustrative embodiments; other formulations may be devised without departing from the spirit and scope of the present invention as defined by the appended claims.

### Example 1—Composition of the Invention

| | Ingredient (INCI name) | % wt/wt | Range |
|---|---|---|---|
| Phase A | Water | QS | |
| | Carbomer | 0.30 | 0.1-0.5 |
| Phase A-1 | Propanediol | 2.00 | 0.5-4.0 |
| Phase B | Avobenzone | 3.0 | 1.0-3.0 |
| | Homosalate | 10.0 | 5.0-15.0 |
| | Octisalate | 5.0 | 1.0-5.0 |
| | Octocrylene | 7.0 | 5.0-10.0 |
| | C₁₂₋₁₅ alkyl benzoate | 5.0 | 2.5-7.5 |
| | Cetearyl alcohol | 1.90 | 1.0-3.0 |
| | Helianthus annuus (Sunflower) | 0.50 | 0.25-1.0 |
| | Seed oil | | |
| | Potassium cetyl phosphate | 0.10 | 0.05-0.25 |
| | Glyceryl stearate (and) PEG- | 0.90 | 0.5-1.0 |
| | 100 stearate | | |
| | Mixture of two, preferably | 0.10 | 0.05-1.0 |
| | three, more preferably, five | | |
| | butters selected from: | | |
| | Shorea robusta seed butter; | | |
| | Elaeis guineensis (palm) | | |
| | butter; Theobroma cacao | | |
| | (cocoa) seed butter; | | |
| | Bassia latifolia seed butter; | | |
| | Mangifera indica (mango) | | |
| | seed butter; Garcinia indica | | |
| | seed butter; Illipe butter; | | |
| | Theobroma grandiflorum seed butter; | | |
| | Astrocaryum murumuru seed butter; | | |
| | and Astrocaryum tucuma seed butter | | |
| Phase C | Aminomethyl Propanol | 0.30 | 0.1-0.5 |
| Phase D | Cyclopentasiloxane | 4.00 | 2.0-5.0 |
| | Hydrated Silica | 3.00 | 1.0-5.0 |
| | Allantoin | 0.05 | 0.05-0.25 |
| Phase E | Water (and) | 3.00 | 1.0-5.0 |
| | Butyrospermum parkii | | |
| | (shea) butter extract (and) | | |
| | dimethicone (and) lecithin (and) | | |
| | phospholipids (and) urea (and) | | |
| | tocopheryl acetate | | |
| | Camellia sinensis leaf extract * | 0.30 | 0.1-1.0 |
| | Osmanthus fragrans flower | 1.00 | 0.1-2.0 |
| | extract ** | | |
| | Lecithin (and) sorbitol (and) | 1.00 | |
| | xanthan gum (and) oleic acid * | | |
| | Preservative | 1.00 | |

* In vehicle of glycerin (and) water
** Extracted in vehicle of water (and) propanediol (and) glycerin

Into the main vessel, add water and begin mixing at high speed to create a vortex. Slowly sprinkle Carbomer into the vortex of the batch; mix until uniform. Heat Phase A to 75-80° C. with mixing, then add Phase A-1 ingredient. Mix until uniform while maintaining temperature at 75-80° C. In a separate vessel, combine Phase B ingredients. Heat Phase B to 75-80° C.; mix until uniform. Slowly add Phase B to Phase A/A-1; mix until uniform, maintaining temperature at 75-80° C. Add Phase C ingredient; mix until uniform. Cool A/A-1/B/C to 45-50° C. with mixing. At this lower temperature, add Phase D ingredients, one at a time, with mixing. Allow A/A-1/B/C/D to cool to 40-45° C.; add Phase E ingredients.

### Example 2—In Vitro Photostability Testing

Sunscreen compositions of the present invention as well as four commercial sunscreens were tested for photostability—change in UVA/UVB of less than 10% after irradiation with 18 MEDs emitted from a solar simulator. The sunscreen active ingredients in each sunscreen in each of the tested sunscreens are summarized below:

| | Sun- | Com- | Com- | Com- | Com- |
|---|---|---|---|---|---|
| | screen | mercial | mercial | mercial | mercial |
| | of In- | Sun- | Sun- | Sun- | Sun- |
| | vention | screen 1 | screen 2 | screen 3 | screen 4 |
| Labeled SPF | 55 | 60 | 55 | 50+ | 50+ |
| Avobenzone | 3 | 3 | 3 | 3 | 0 |
| Homosalate | 10 | 15 | 10 | 10 | 0 |
| Octisalate | 5 | 5 | 5 | 5 | 0 |
| Octocrylene | 7 | 7 | 2.8 | 2.7 | 3 |
| Oxybenzone | 0 | 0 | 6 | 6 | 0 |
| Octinoxate | 0 | 0 | 0 | 0 | 7.4 |
| Titanium | 0 | 0 | 0 | 0 | 1.4 |
| Dioxide | | | | | |
| Zinc Oxide | 0 | 0 | 0 | 0 | 16.4 |

All three of the non-mineral commercial sunscreens (i.e., not containing zinc oxide or titanium dioxide) contained SPF Boosters—ingredients that increase the SPF of the product.

Commercial Sunscreen 1 contained three (3) SPF Boosters: butyl octyl salicylate (“BOS”) sold under the tradename HALLBRITE® BHB HALLBRITE® by The Hallstar Company (Chicago, Ill.); styrene/acrylates copolymer sold under the tradename SUNSPHERES® Powder by the Dow Chemical Company (Midland, Mich.); and diethylhexyl syringylidenemalonate available under the tradename OXYNEX® ST from EMD Performance Materials Corp. (Philadelphia, Pa.).

Commercial Sunscreen 2 contained three (3) SPF Boosters: BOS, SUNSPHERES Powder; and diethylhexyl 2, 6-naphthalate (“DEHN”) sold under the tradename CORAPAN® TQ by Symrise, Inc. (Teterboro, N.J.).

Commercial Sunscreen 3 contained one (1) SPF Booster, BOS.

Commercial Sunscreen 4 contained zero (0) SPF Boosters.

Together (i) butyl octyl salicylate, (ii) diethylhexyl 2, 6-naphthalate and (iii) diethylhexyl syringylidenemalonate are referred to as “SPF Boosting Esters”.

In certain preferred embodiments, the broad-spectrum sunscreen of the present invention does not any contain SPF Boosting Esters.

Each sunscreen was applied at a concentration of 2 mg/cm² to VITRO-SKIN®. The ratio of UVA/UVB absorption was calculated prior to irradiation and after irradiation with a dose equivalent to 18 MEDs. All five sunscreens were determined to be photostable as that term is defined in the present application.

| | Sun- | Com- | Com- | Com- | Com- |
|---|---|---|---|---|---|
| | screen | mercial | mercial | mercial | mercial |
| UVA/UVB | of In- | Sun- | Sun- | Sun- | Sun- |
| Ratio | vention | screen 1 | screen 2 | screen 3 | screen 4 |
| Before | 0.88 | 0.88 | 0.95 | 0.82 | 0.81 |
| irradiation | | | | | |
| After dose of | 0.83 | 0.91 | 0.96 | 0.79 | 0.85 |
| 18 MEDs | | | | | |

1. A broad-spectrum, sunscreen composition having an SPF of at least 30, and containing
(a) avobenzone;
(b) octrocrylene;
(c) a synergistic anti-glycation complex consisting essentially of
(i) an extract of a botanical ingredient in the genus Osmanthus,
(ii) an extract of Camellia sinensis; and
(iii) optionally, Butyrospermum parkii (shea) butter.

2. The broad-spectrum, sunscreen composition of claim 1 wherein the extract of a botanical ingredient in the genus Osmanthus is Osmanthus fragrans.

3. The broad-spectrum, sunscreen composition of claim 2 wherein synergistic anti-glycation complex consists of (i) an extract of Osmanthus fragrans, (ii) an extract of Camellia sinensis and (iii) Butyrospermum parkii (shea) butter.

4. The broad-spectrum, sunscreen composition claim 3 wherein the sunscreen composition further contains at least one salicylate derivative that absorbs ultraviolet radiation in the range 290-320 nm.

5. The broad-spectrum, sunscreen composition of claim 4 wherein the at least one salicylate derivative that absorbs ultraviolet radiation in the range 290-320 nm is homosalate and/or octisalate.

6. The broad-spectrum, sunscreen composition of claim 5 wherein the sunscreen composition contains homosalate and octisalate in a ratio of about 2:1.

7. The broad-spectrum, sunscreen composition of claim 6 wherein the ratio of salicylate derivatives to octocrylene is at least 2:1.

8. The broad-spectrum, sunscreen composition of claim 7 wherein the broad-spectrum, sunscreen composition is further comprised of at least two butters (in addition to Shea Butter) selected from the group consisting of Shorea Robusta seed butter; Elaeis guineensis (palm) butter; Theobroma cacao (cocoa) seed butter; Bassialatifolia seed butter; Mangifera indica (mango) seed butter; Garcinia indica seed butter; Illipe butter; Theobroma grandiflorum seed butter; Astrocaryum murumuru seed butter; and Astrocaryum tucuma seed butter.

9. The broad-spectrum, sunscreen composition of claim 8 wherein the broad-spectrum, sunscreen composition is further comprised of an antioxidant vitamin.

10. The broad-spectrum, sunscreen composition of claim 9 wherein the antioxidant vitamin is vitamin E or a derivative thereof.

11. A broad-spectrum, sunscreen composition according to claim 10 that does not contain oxybenzone.

12. A broad-spectrum, sunscreen composition according to claim 11 that does not contain octinoxate.

13. A broad-spectrum, sunscreen composition according to claim 12 that does not contain an SPF boosting ester selected from the group of butyl octyl salicylate, diethylhexyl 2, 6-naphthalate and diethylhexyl syringylidenemalonate.

14. A broad-spectrum, sunscreen composition having an SPF of at least 30, and containing
(a) at least one mineral sunscreen selected from the group of titanium dioxide and zinc oxide
(b) a synergistic anti-glycation complex consisting essentially of an extract of a botanical ingredient in the genus Osmanthus and an extract of Camellia sinensis; and, optionally, Butyrospermum parkii (shea) butter.

15. A broad-spectrum, sunscreen composition according to claim 14, having an SPF of at least 50.
